(19) United States
(12) Patent Application Publication
(10) Pub. No.: US 2023/0316947 A1
(43) Pub. Date: Oct. 5, 2023

(54) SYSTEMS AND METHOD FOR MEASURING ATTENTION QUOTIENT

(71) Applicant: MINDWELL LABS INC., New York, NY (US)

(72) Inventors: Shaun S. NANAVATI, New York, NY (US); Saurabh KANAUJIA, New York, NY (US)

(73) Assignee: MINDWELL LABS INC., New York, NY (US)

(21) Appl. No.: 18/023,157

(22) PCT Filed: Aug. 27, 2021
(86) PCT No.: PCT/US2021/047995

§ 371 Date: Feb. 24, 2023

Related U.S. Application Data
(60) Provisional application No. 63/071,608, filed on Aug. 28, 2020.

(51) Int. Cl. G09B 19/00; A61B 5/16; A61B 5/024

(52) U.S. Cl. CPC G09B 19/00; A61B 5/168; A61B 5/02438

(57) ABSTRACT

Disclosed are systems and methods for measuring and monitoring attention of a user and may determine an attention quotient (AQ). In some examples, these systems and methods may calculate a user's AQ based on variation in heart rate while the user's eyes are open and closed. The system may provide content and programming to the user based on their AQ

### CROSS-REFERENCE TO RELATED APPLICATIONS

This application claims benefit under 35 U.S.C. § 119(e) of U.S. Provisional Application No. 63/071,608, filed Aug. 28, 2020, content of which is incorporated herein by reference in its entirety.

### FIELD

The present invention is directed to systems and method for measuring attention of a user.

### BACKGROUND

The following description includes information that may be useful in understanding the present invention. It is not an admission that any of the information provided herein is prior art or relevant to the presently claimed invention, or that any publication specifically or implicitly referenced is prior art.

The most widely used medical measure of attention is an attentional battery, such as the D-KEFS, which includes several specific tests of attention. This standard can be extremely time-consuming (10+ hours including report) and expensive ($10k+). It also requires a specialist (neuropsychologist) to administer it.

### SUMMARY

Disclosed are systems and methods for measuring and improving a person's attention using a new metric, called Attention Quotient (“AQ”). AQ may be a composite score which combines various different measures into that are based upon cardiovascular biomarkers, respiratory biomarkers and/or answers to self-reported questions. The disclosed technology may automatically deliver programs through an application that improve attention and mindfulness that are personalized for each user based on their AQ score.

Human attention is a central cognitive process, perhaps even more important than intelligence, for success in various life activities. Current approaches in measuring attention depend upon self-reporting which is vulnerable to self-deception and bias. The disclosed technology for measuring AQ allows for a non-invasive physiological measure of attention that can then be utilized for tailored intervention to improve attention and thereby produce maximum benefits for an individual user. Additionally, disclosed are systems and methods for performing an analysis of attention based on cardiovascular biomarkers into its composite aspects, creating a precise “drill-down” profile of individual attentional qualities.

Accordingly, the disclosed technology may be provided to users via a mobile app that may be self-administered. Users can measure their attention at any time and at any place without requiring any third parties to administer an AQ test. Accordingly, attention may be measured by the user multiple times and therefore longitudinal and dynamic attention data may be recorded and determined, including in response to providing attention tasks to monitor improvement. Accordingly, the disclosed systems and methods could automatically deliver personalized content and programs to improve their attention based on data regarding past changes in attention after delivery of programming.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 depicts an example of an overview of a system for implementing the disclosed technology.

FIG. 2 depicts a flow chart showing example processes for implementing the disclosed technology.

FIG. 3 depicts a flow chart showing an example process for implementing the disclosed technology.

FIG. 4 depicts a flow chart showing an example process for implementing an attention quotient test.

FIGS. 5-9 are bar graphs showing performance on tests of attention based on Q1 Scores (FIG. 5), Q2 Scores (FIG. 6), Q3 Scores (FIG. 7), Q4 Scores (FIG. 8) and Total AQ™ scores.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Systems

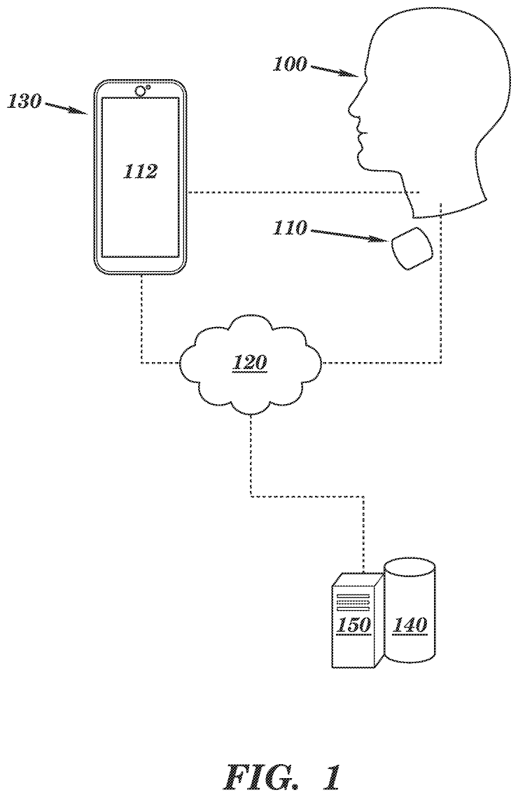

FIG. 1 illustrates an example system for implementing the disclosed technology. For instance, the system may contain a computing device 130 with a display 112, a network 120, a patient 100, a sensor 110, a server 150, and database 140. The computing device 130 may be any suitable computing device, including a computer, laptop, mobile phone, etc. The network 120 may be wired, wireless, or various combinations of wired and wireless. The server 150 and database may be local, remote, and may be combinations of servers 150 and databases 140, or could be local processors and memory.

The sensor 110 may be a smart phone, smart watch, smart ankle bracelet, smart glasses, smart ring, patch, band, or other device that suitably could be retained on the patient 100 and output heart rate data from the patient. In other examples, the wearable 110 may be a clinical grade ECG system. In some examples, the sensor 11 may be a camera on a mobile device and may record the heart rate data by fluctuation in colors of the capillaries detected by the camera.

### Attention Monitoring and Training System

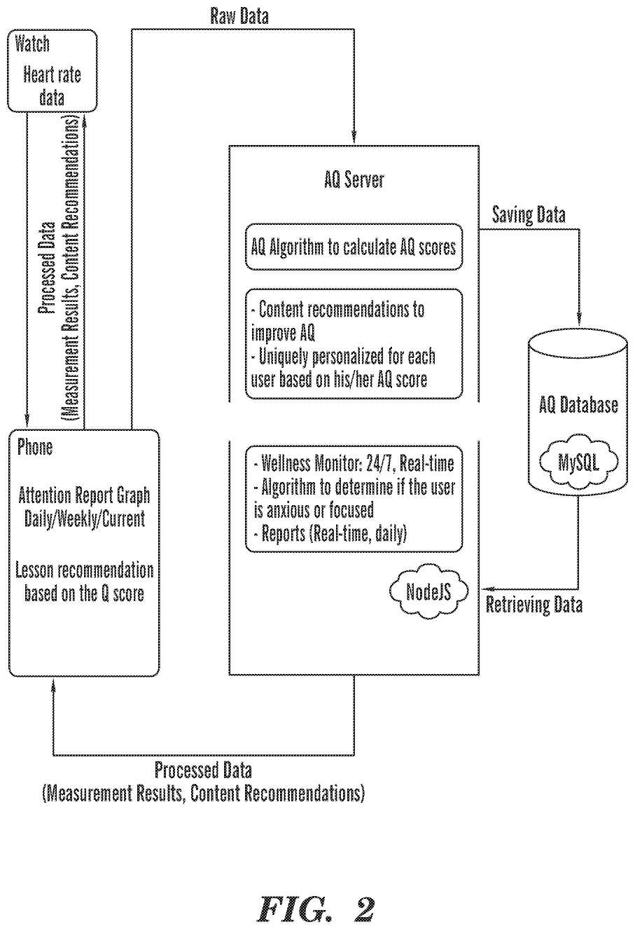

FIG. 2 illustrates an overview of an example system for monitoring attention and delivering programming to the user to improve attention. Accordingly, as illustrated, a server may execute various algorithms and models utilized to determine AQ, and send content recommendations to improve AQ. These content recommendations may be uniquely personalized for each user based on their AQ scores as described herein.

The AQ may be determined from heart rate data. The heart rate data may be output from a smart watch with ECG capabilities or from a smartphone with camera and sent to a user's mobile device/smartphone. The user's mobile device may include an application that may provide content recommended for the user to improve their AQ and a reports showing the user's AQ—including the current AQ and trends of the AQ over time (e.g. daily, weekly, monthly).

In some examples, raw heart rate data may be processed on a user's smart watch, on a user's smart phone/mobile device, or may be sent in raw format to a server where the AQ algorithms are stored for processing. A server may include a database connected to the server that includes AQ data stored from the user and third party users.

In some examples, raw respiratory data may be processed on a user's smart watch, on a user's smart phone/mobile device, or may be sent in raw format to a server where the AQ algorithms are stored for processing. A server may include a database connected to the server that includes AQ data stored from the user and third party users.

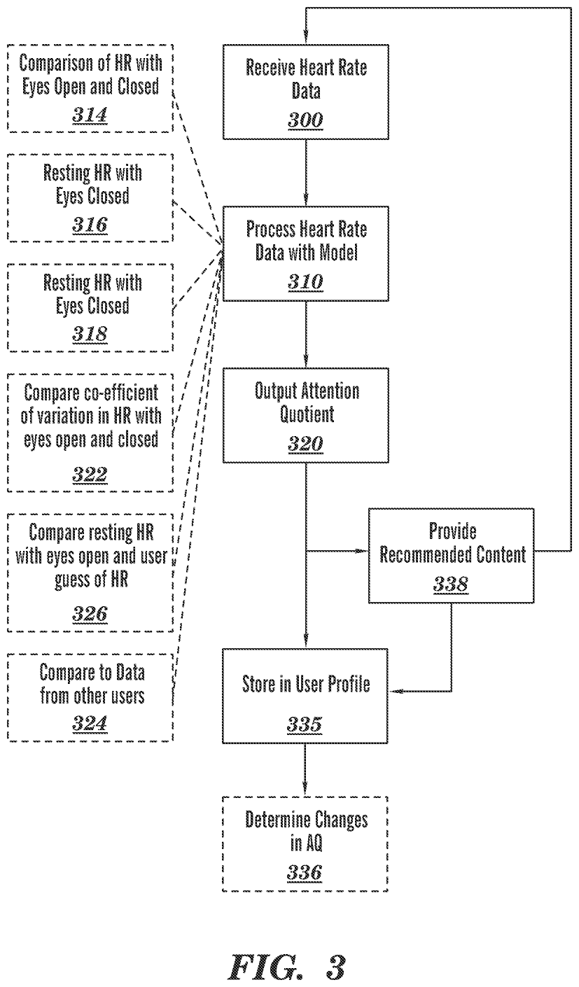

FIG. 3 illustrates an example process for determining AQ and delivery content to a user. For instance, heart rate data may be received 300 from a smart watch, or a mobile device with a camera after a user presses their finger over the camera sensor. Then, the heart rate data may be processed with a model 310 to determine various components of AQ.

For instance, the model may perform comparison of heart rate data with the user's eyes open, eyes closed, and with other users of the technology. The measures may include: (1) comparisons of the heart rate with the eyes open and the eyes closed 314, (2) resting heart rate with the eyes closed 316 and (3) with the eyes open 318, (4) the co-efficient of variation of the heart rate with the eyes open and the eyes closed 324, (5) comparison of the resting heart rate with eyes open and the user's guess of the resting heart rate, and (6) data from other users.

Then, various of these metrics may be combined to output an attention quotient 320. In some examples, the attention quotient may be a statistical average or combination of these metrics.

Next, the system may store the attention quotient and details in a user profile 335 associated with the user. This may include a date and time stamp for the attention quotient and any other contextual information including day of the week. In some examples, the system may store multiple attention quotients for a user and then determine changes in AQ 336, which may include trends or associations with other contextual information.

After determining the AQ, the system may then provide recommended content to the user 338 to improve the AQ generally, or to improve a component factor of the AQ. The content may include training lessons, meditations, breathing exercise, physical movements, sound therapy, change in user's environment (room temperature and lighting), etc. In some examples, every time the user completes content delivered to the user through the system, the system may store the content session completion information in the user profile 335.

Accordingly, the system may provide recommended content based on changes in AQ, or specific components/measures of AQ. In some examples, the system may determine which component/measure of AQ needs to improve the most and which content will be most helpful to a specific user to improve that component.

### Attention Quotient Measurement Application

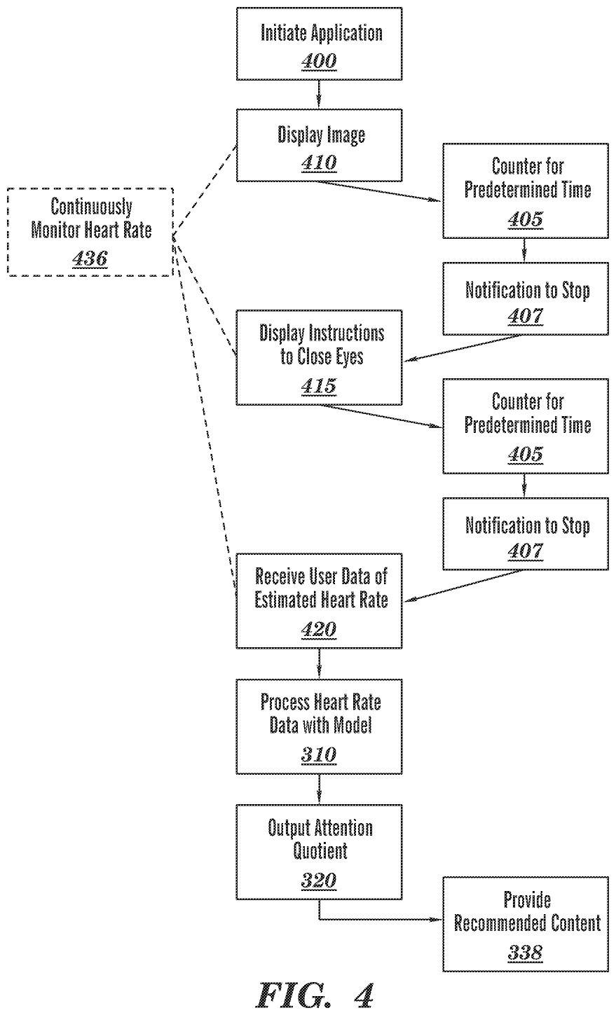

The disclosed technology, in some examples, measures AQ by looking at various cardiovascular data, for instance changes in heart rate data during a short test (e.g. 1, 2, 3, 4, 5, 6, 7, 8, 9, 10, or other suitable duration test). FIG. 4 illustrates a flow chart showing an example process for administering a test to measure AQ. The test may measure a user's heart rate continuously 436 while their eyes are open and their eyes are closed, and utilize the data to determine attention quotient metrics that estimate a user's attention.

Following is one example implementation of such a test that may be implemented by the disclosed technology. However, while steps are implemented in an example order, certain steps may be performed in different orders and remain effective. In some examples, only certain of the steps may be necessary to measure the AQ. For instance, in some examples, the system may not request the user's subjective estimate of heart rate.

In one example, the system will initiate an application 400, which may be an application on a mobile device 130 and/or smart watch. Then, in some examples, the system may display an image 410 on a display 112 of the mobile device 130 for a first phase of the test. In some examples, this may be a calming image, for instance a rotating globe or other relatively slowly moving, relaxing image. The system may provide instructions to the user 100 through an interface to maintain their focus on the image displayed on the display 112 for the first phase.

Then, a counter/timer may be initiated allows a predetermined amount of time 405 to elapse during the first phase. In some examples, the time may be 20, 30, 40, 50, 60, 70, 80, 90, 100, 110, 120, 130, 140, 150, 160, 170, 180 seconds, or other suitable durations. During the first phase, the system may continuously measure the user's heart rate 436. For instance, in some examples, the heart rate may be captured ever 1, 2, 3, 4, 5, 6, 7, 8, 9, or 10 seconds and stored in a local memory or sent to a server.

Once the counter for the predetermined time elapses 405, the system may provide a notification of the user to stop 407, which may include energizing a vibrating element, a visual instruction to step on the display 112, and audio indication to stop emitted through a speaker, or simply stopping the graphical representation of the calming image and/or video. Accordingly, this will signal the end of the first phase of the test.

Next, the system may display instructions on the display 112 (or provide audio instructions) for the user to close their eyes 415 to initiate the second phase of the test. The system may similarity initiate a counter/timer for a predetermine time 405 for the second phase, which in some examples, may be 20, 30, 40, 50, 60, 70, 80, 90, 100, 110, 120, 130, 140, 150, 160, 170, 180 seconds, or other suitable durations.

Once the counter for the predetermined time elapses 405, the system may provide a notification of the user to stop 407, which may include energizing a vibrating element or providing an audio indication to stop through a speaker in the case of the second phase where the user's eyes are closed.

Lastly, the system may request the user provide an estimate of their own heart rate 420 through a user interface. This step may be performed at various times, and an actual heart rate 436 may be recorded at the same time the user provides the estimate.

Accordingly, the system may the process the heart rate data with a model 310 to output an attention quotient 320 as described herein. Additionally, the application may then deliver the content/programming 338 to the user's smart device (e.g. mobile device or watch). The content/programming may include training lessons, meditations, breathing exercise, physical movements, sound therapy, change in user's environment (room temperature and lighting), etc.

In some examples, the content/programming may be delivered through the application so that the system may store and track the user's engagement and usage of specific content and programs. Accordingly, the application may evaluate the impact on each person, as measured by changes in the user's AQ scores. Such learning allows our system to create and store a unique profile of each user, thus enabling improvements in ongoing recommendations that can be uniquely and precisely targeted in order to improve that person's attentional states.

In some examples, accuracy of heart rate variability, such as vagal tone can be improved by incorporating various respiratory data. It is noted that vagal tone can be used as an additional input in calculating AQ.

The disclosed technology, in some examples, measures AQ by looking at various combinations of cardiovascular data and respiratory data.

### Algorithms for Determining Attention Quotient

In some examples, the system may utilize various algorithms to determine the AQ from heart rate data as disclosure herein. In some examples, this may include determining various components that may be combined to form a single score or value of an AQ.

Following are examples of five “quotients” that may be determined. The first quotient “Q1—Awareness” may be related to awareness. In some examples, it may be calculating with the following steps:
measure user's co-efficient of variation (“COV”) of heart rate captured while eyes open;
compare that measure with all other users (group analysis) and rank from distance to the mean; and
provide the Q1 score.

The second quotient “Q2—Rest” may be related to rest. In some examples, it may be calculating with the following steps:
measure user's COV of heart rate captured while eyes closed;
compare that measure with all other users (group analysis) and rank from distance to the mean; and
provide the Q2 score.

The third quotient “Q3—Introspection” may be related to introspection. In some examples, it may be calculating with the following steps:
measure user's difference in average heart rate captured while their eyes are open and while their eyes are closed;
compare that measure with all other users (group analysis) and rank from distance to the mean; and
provide the Q3 score.

The fourth quotient “Q4—Calm” may be related to anxiety. In some examples, it may be calculating with the following steps:
measure user's difference in COV captured while eyes are open and while their eyes are closed;
compare that measure with all other users (group analysis) and rank from distance to the mean; and
provide the Q4 score.

The fifth quotient “Q5—Somatics” may be related to somatics. In some examples, it may be calculating with the following steps:
measure user's difference in average heart rate while eyes open and the user's subjective guess as to their average heart rate;
compare that measure with all other users (group analysis) and rank from distance to the mean; and
provide the Q5 score

Calculate AQ. Lastly, the system may calculate a composite score and output an attention quotient. In some examples, it will be performed with the following steps, but may be performed with similar or alternative statistical analysis techniques:
take weighted average of all the five Qs, or less than all five Qs, utilizing Cronbach Alpha as diagnostic guide for reliability in some examples;
exclude any missing data from any Qs and adjust weighted average accordingly; and
exclude any redundant data (multiple tests for single user).

It should be understood that AQ can be calculated by taking weighted average of all the five Qs, or less than all five Qs. For example, AQ can be calculated by taking weighted average of only four of the Qs, only three of the Qs or only two of the Qs. In some non-limiting examples, AQ can be calculated by taking weighted average of Q1-Q4.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### Example

Even a cursory analysis of the processes of attention will reveal multiple neurological and attentional systems at play. Our experience of attending to something is obviously more complex than it might seem at first glance. Even within the domain of psychology many different measures exist to determine specific qualities of our attentional process. Generally speaking, the fields of neuropsychology, clinical psychology, cognitive psychology, and psychometric testing all define attention differently and utilize very different methods to make measurements.

AQ™ (Attention Quotient): AQ™ stands for Attention Quotient, much like IQ stands for Intelligence Quotient or EQ represents Emotional Quotient. AQ™, just like IQ, is comprised of several sub-components. Whereas IQ is based on person-to-person testing, AQ™ is calculated based on cardiac parameters. The components of AQ™ and their derivation are as follows:
1. Awareness (Q1): Awareness is the co-efficient of variation of heart rate during the 2-minute Eyes Open task.
2. Rest (Q2): Rest is the co-efficient of variation of heart rate during the 2-minute Eyes Closed task.
3. Introspection (Q3): Introspection is the difference in absolute heart rate between the Eyes Open and Eyes Closed conditions.
4. Calm (Q4): Calm is the difference in the co-efficient of variation between the Eyes Open and Eyes Closed conditions.

Each sub-component is then turned into a Q or quotient by normalizing the individual score (subtracting the individual user score from the group average and dividing by the standard deviation of the group, then multiplying the result by 15 and then adding 100).

### Method

Design: This study used both within and between-subjects analyses to examine changes in heart rate and vagal activity with increasing cognitive complexity. Measures included mindfulness (Kentucky Inventory of Mindfulness [KIM]; Baer et al., 2004), cognitive bias (Frederick, 2005), working memory (Digit Span Forward-and-Backward; Drozdick et al., 2012), and a 4-minute baseline EKG from which various cardiac parameters were derived. These parameters form the basis of the Attention Quotient (AQ™) measurement. The Tower of Hanoi task was then administered over three trials while measuring autonomic activity. The last trial in this study is a unique seven-disc challenge designed to identify expert performance on the task.

Participants: Nineteen adults (11 women, 8 men) between the ages 25 and 60, were recruited from a workshop at the MENLA Retreat Center in Phoencia, NY. Written informed consent was obtained from all participants. No participants were excluded from the study. Tower performance was not recorded for one participant.

### Materials and Measures

Self-Report Measures: The KIM (Baer et al., 2004) is a 39-item self-report questionnaire which demonstrates good internal consistency (Baum et al., 2009), with previous alpha scores of 0.91, 0.84, 0.76, and 0.87 for the respective subscales of Observe, Describe, Act, and Accept, and adequate to good test-retest reliability (Baer et al., 2004). The KIM assesses mindfulness along four subscales: Observing, Labeling, Acting With Awareness, and Acceptance Without Judgment. Items commonly reported in the subjective experience of mindfulness are presented on a 5-point Likert-type scale. For example, one item states “I intentionally stay aware of my feelings” with five choices for participants (1=never or very rarely, 3=sometimes true, 5=very often or always true). Carmody and Baer (2008) reported all four scales were sensitive to change in a group of people with chronic health problems. While internal consistency is reported as adequate to good (Nunnelly, 2008), it is unknown if the strength of the measure remains when given to different clinical samples (Baum et al., 2009).

### Executive Function Measures:

The Tower. The Tower of Hanoi is a non-verbal test of executive function (Fine & Delis, 2011). In the task, an individual is asked to move a tower of discs from the first pole to the third. The participant is instructed that they are not allowed to place a big piece on top of a little piece, nor move more than one disc at a time. This task requires various underlying cognitive skills to be used together, namely those of inhibition, planning, and working memory. Tower performance is a quotient calculated by taking the number of moves by the subject to solve the task and dividing it by the minimum number of moves required. For example, if a participant were to solve the four-disc task (15 move minimum) within 30 moves, a score of 2 would be assigned. If the subject cannot solve the task, a maximum score of 5 is assigned. For example, in the four-disc task, 75 moves would warrant a score of 5. Three versions were provided: a four-disc version (15 move minimum), a five-disc version (31 move minimum), and a seven-disc version (127 move minimum). A previous study (Welsh et al., 1991) found performance on the four-, five-, and six-disc met normative cognitive development at 6 years of age, 10 years of age, and adolescence respectively. Since this study took physiological measures while the subject was performing the task, an autonomic signature of superior performance was sought. Internal consistency for the total achievement score in the Tower of Hanoi task used in the Delis-Kaplan Test of Executive Function (Fine & Delis, 2011) with four- and five-disc trials, was found to be marginal (0.60-0.69) with low test-retest reliability (≤0.59). In terms of validity, Strauss et al. (2006) call for additional factor-analytic study.

Digit Span: The Digit Span Forward/Backward (WAIS-IV-R; Drozdick et al., 2012) is a subtest from the standard intelligence test (IQ) that is delivered aurally to assess working memory. The participant is asked to memorize a string of numbers and repeat them back in three different sets: the set forward, the set backward, and rearranging the numbers of the set in sequential order. Each set contains 16 strings of numbers, starting with two digits and increasing by one digit after every two trials. If the participant is unable to repeat both strings in a set, the strings are no longer administered. The score is derived from the number of strings that the participant was able to properly repeat back. A separate score is determined for each set (maximum of 16), which is reported along with a score of the responses combined (maximum of 48). Drozdick et al. (2012) reports solid reliability and validity for the Working Memory Index and Digit Span subtest in the Technical Manual. Digit span was also found to have a 0.69 loading with General Ability (g) in the fourth edition (Lichtenberg & Kaufman, 2009).

Physiological Instruments: The parasympathetic nervous system is a branch of the autonomic nervous system considered to be a component of the physiological substrate of attentional processes (Porges, 1992, 1994; Pribram & McGuinness, 1972) and working memory (Hansen et al., 2003). In this study, changes in the parasympathetic nervous system were considered the primary outcome variable during tasks of increasing complexity in the Tower of Hanoi task. The EKG data was collected using a Nightingale PPM2 monitor attached to an Acer laptop running a commercially available system for analysis of ANS activity (ANX 3.0 Autonomic Monitor by ANSAR Technologies). During each condition, recordings were made at a 250-Hz sampling frequency and stored on the laptop hard drive. A measure of parasympathetic tone (RMSSD) was derived through analysis of 480 readings of the Heart Beat Interval (IBI).

Procedure: After obtaining informed consent, participants were asked to sit in an upright chair (back at a 90-degree angle). Basic demographic information was first taken, including age, gender, handedness, ethnicity, medical history, and recent consumption of stimulants (i.e., medications or coffee). This was followed by the KIM and Digit Span tasks. This section of the procedure took approximately 10 minutes.

After initial trait measures were taken, EKG data were recorded with Ag/AgCL electrodes positioned in the standard X, Y, and Z lead positions (two leads just below the clavicles and a ground just under the ribcage on the left side). Participants were instructed to relax and refrain from unnecessary movement or speech to control for the artifacts of motion or speech. Baseline measures with the eyes open for 2 minutes and then with the eyes closed for 2 minutes were taken. This was followed by the three Tower of Hanoi trials in increasing difficulty consisting of a four-disc version (easy), a five-disc version (medium), and a seven-disc version (difficult).

Each trial lasted as long as it took for the subject to either solve the Tower of Hanoi task or give up. The first trial lasted roughly 1 to 2 minutes, the second 5 to 10, and the last trial lasted 8 to 12 minutes. The task was terminated if total number of moves equaled 6 times the minimum number of moves for that particular trial. After the last trial of the Tower of Hanoi, participants were asked to remove the leads. This was followed by the Stroop test. Finally, the subject was then asked open-ended questions to discuss their experience and to debrief about the purpose of the task. An individual autonomic test report was printed immediately and provided to the subject upon request.

Data Analysis: Raw signals from two electrodes (and one ground) were used to produce an IBI data set from which frequency domain was then derived using ANSAR software, providing a measure of RFA, LFA, and balance between the two systems. A correlation matrix was created to examine relationships between twelve variables: AQ™-Awareness, KIMS (and sub-scales), Digit Span (and sub-scales), Cognitive Bias, and Tower performance. High and low quartile segments of each score were derived. High and low AQ™ scores were correlated against all high and low scores on each test.

Hypothesis: We hypothesized that the high and low quartiles for AQ™ will correlate with the high and low scores on all tests of attention. Furthermore, we hypothesized that a significant moderated regression for AQ™ will exist that unites the differing aspects of attention into a single, unified equation for attention.

### Results

These results compared participants with high and low scores in each Q and the final weighted AQ™ on their attentional performance on the tests for mindfulness, problem-solving and memory. High Q and AQ™ scores are based upon the top twenty percent of participants. Low scores are the lowest twenty percent of scores. Scores of mindfulness are based upon the self-report of the Kentucky Inventory of Mindfulness Skills (KIMS). Problem-solving is based upon performance in the second trial of the neuropsychological task, the Tower of Hanoi. Memory is defined by the overall score in the digit span test from the Wechsler Intelligence Test.

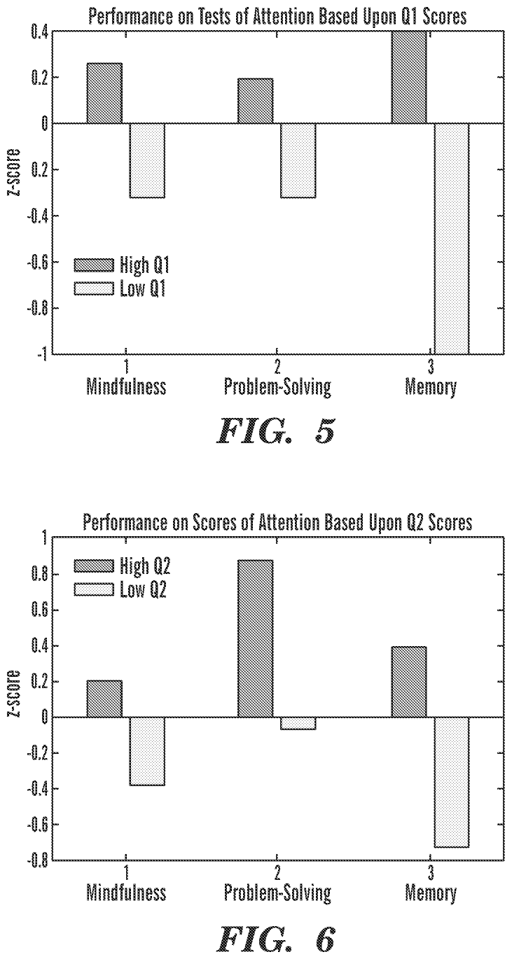

Q1 (Awareness): As seen from FIG. 5, for those with a higher Q1 score, all scores of attentional performance were higher than those with low Q1 scores. Q1 is derived from the co-efficient of variation during a two minute eyes closed task.

Q2 (Rest): As seen from FIG. 6, for those with a high Q2 score, all scores of attentional performance were also higher than those with low Q2 scores. Q2 is derived from the co-efficient of variation during a two minute eyes closed task.

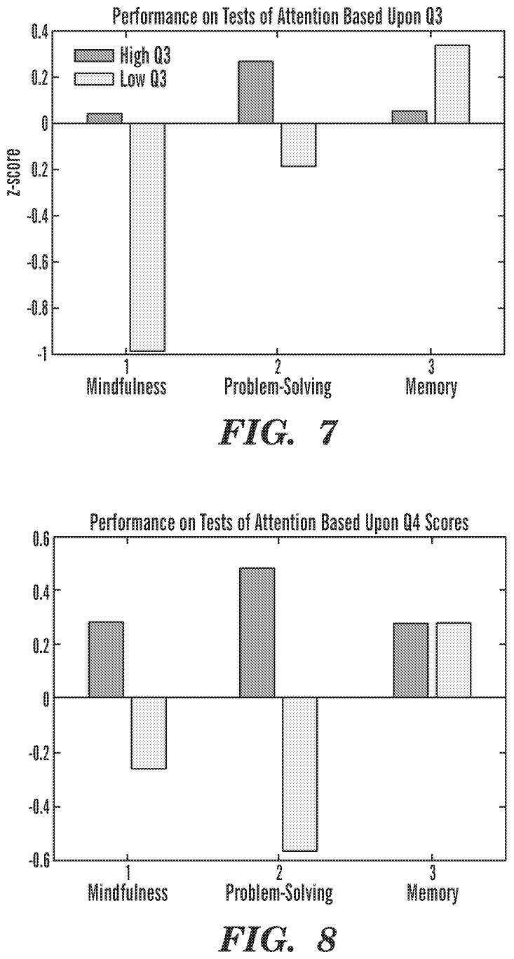

Q3 (Introspection): As seen from FIG. 7, for those with higher Q3 scores, tests of mindfulness and planning were higher than those with lower scores. Memory, however, was reversed: those with low Q3 scores demonstrated higher digit span scores than those with lower digit span scores. Q3 is derived from the difference in average heart rate between the two-minute eyes open and two-minute eyes closed conditions.

Q4 (Calm): As seen from FIG. 8, for those with higher Q4 scores, tests of mindfulness and planning were higher than those with lower scores. Scores of memory were equal among those with high and low Q4 scores.

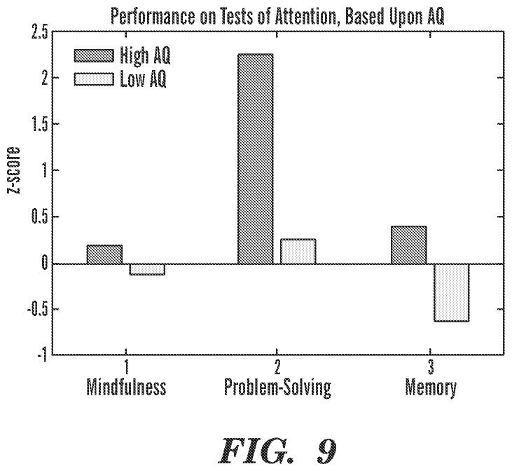

Total AQ™: In FIG. 9, the results are broken up by those with high and low AQ™ scores. High AQ™ is defined as above 125. Low AQ™ scores are those with scores under 95.

### DISCUSSION

These results provide some interesting findings which suggest that the heart plays a much larger role in cognitive processes such as attention than previously considered. The heart and brain are evidently integrated within a single autonomic network and, thus, must work in symphony for maximum mental well-being.

The AQ™ metric categories cover three different domains within the psychological portrait of an individual: problem-solving, memory, and mindfulness (personality). This measure puts mental health on firm physiological ground, providing a framework for an etiological and truly scientific psychology. AQ™ provides a single number by which to measure individual attentional quality, which includes both the states and traits of the individual.

While these measures are calculated based on basic heart rate, the individual Qs can incorporate a variety of cardiovascular and autonomic inputs for AQ™. This includes varying measures of parasympathetic activity, such as rmSDD, and other predictors of general health such as pNN50. The protocol provides an experimental situation and system by which rapid mental check-ups can be conducted remotely with minimally invasive interactions, using heart rate and any of its autonomic derivations.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A system for monitoring attention, the system comprising:
a sensor for outputting heart rate data;
a memory containing machine readable medium comprising machine executable code having stored thereon instructions for performing a method;
a control system coupled to the memory comprising one or more processors, the control system configured to execute the machine executable code to cause the control system to:
receive a set of heart rate data from a user output from the sensor;
process the set of heart rate data to output an attention quotient (AQ); and
store the attention quotient referenced to a unique identifier referenced to the user in the memory.

2. The system of claim 1, wherein the control system is configured to execute the machine executable code to further cause the control system to send a notification to a user interface associated with the user with a set of content based on the attention quotient.

3. The system of claim 1, wherein the process the set of heart rate data comprises at least one of: a comparison of a first heart rate with the user's eyes open and a second heart rate with the user's eyes closed, a comparison of the first and second heart rates with heart rate data from additional users, a difference in coefficient of variation between the first and second heart rate, and a difference between the first heart rate and an estimated heart rate received from a user interface associated with the user.

4. The system of claim 1, wherein the attention quotient is an estimate of at least one of awareness, rest, introspection, anxiety, or somatics.

5. The system of claim 1, wherein the set of heart rate data is continuously acquired from the sensor during administration of a test.

6. The system of claim 5, wherein the control system is configured to execute the machine executable code to further cause the control system to send a notification to an interface associated with the user for the user to open and close their eyes at various times during the test.

7. The system of claim 5, wherein the test is one, two, three, or four minutes in duration.

8. The system of claim 2, wherein the set of content comprises at least one of: training lessons, meditations, breathing exercise, physical movements, sound therapy, or changes in the patient's room temperature or lighting.

9. The system of claim 8, wherein the control system is configured to execute the machine executable code to further cause the control system to:
receive a second set of heart rate data after delivery the set of content;
process the second set of heart rate data to output a second AQ;
perform a comparison between the AQ and second AQ; and
send a second notification to the user interface associated with the patient with content based on the comparison.

10. The system of claim 8, wherein the control system is configured to execute the machine executable code to further cause the control system to store to the memory a number and type of the set of content performed through the interface by the user.

11. The system of claim 1, wherein the sensor is an ECG sensor on a smart watch.

12. The system of claim 1, wherein the sensor is a camera on a mobile device.

13. The system of claim 1, wherein the control system is configured to execute the machine executable code to further cause the control system to receive a second set heart rate data from a second sensor associated with a second user.
